(19) United States
(12) Patent Application Publication
(10) Pub. No.: US 2020/0330734 A1
(43) Pub. Date: Oct. 22, 2020

(54) MEDICAL TUBE

(71) Applicant: ASAHI INTECC CO., LTD., Seto-shi (JP)

(72) Inventors: Shuji SUGITA, Seto-shi (JP); Akira SAWAI, Seto-shi (JP)

(73) Assignee: ASAHI INTECC CO., LTD., Seto-shi (JP)

(21) Appl. No.: 16/918,471

(22) Filed: Jul. 1, 2020

Related U.S. Application Data
(63) Continuation of application No. PCT/JP2018/017886, filed on May 9, 2018.

(51) Int. Cl. A61M 25/09; A61M 25/01; A61M 25/00

(52) U.S. Cl. CPC A61M 25/09; A61M 2025/0002; A61M 25/0054; A61M 25/0138

(57) ABSTRACT

A medical tube includes an inner tube shaft which has a proximal end portion and a distal end portion formed at a distal end side of the proximal end portion. The distal end portion has an outer diameter that is smaller than an outer diameter of the proximal end portion, and has on a surface thereof a slit extending in a first direction with respect to a longitudinal direction of the medical tube. The medical tube also includes an outer tube shaft that covers an outer periphery of the distal end portion of the inner tube shaft and has on a surface thereof a slit extending in a second direction crossing the first direction.

### CROSS REFERENCE TO RELATED APPLICATION

This is a Continuation of PCT/JP2018/017886 filed May 9, 2018. The disclosure of the prior application is hereby incorporated by reference herein in its entirety.

### BACKGROUND

The disclosed embodiments relate to a medical device. Specifically, the disclosed embodiments relate to a medical tube for use in medical fields.

Traditionally, various guide wires have been proposed for guiding a catheter and the like which is inserted, when used, into a tubular organ such as a blood vessel, gastrointestinal tract, and ureter, or body tissues for the purposes of treatment and laboratory tests. Guide wires usually require blood-vessel followability for allowing them to follow along a curved blood vessel of a patient; torquability for allowing the distal ends of the guide wires to rotate smoothly inside a blood vessel of a patient; and further distal-end flexibility for preventing damage to a blood vessel of a patient.

Among them, a guide wire with a pressure sensor for measuring the internal pressure of a blood vessel of a patient needs to be configured to have a lumen through which the pressure sensor and a lead wire therefor are passed, and thus its hollow tubular structure is particularly required to satisfy the aforementioned requirements.

For example, Japanese Patent No. 5866371 describes a pressure-sensor guide wire having a pressure sensor for measuring the internal pressure of a blood vessel of a patient (see FIG. 1 and others).

The pressure-sensor guide wire 10 described in Japanese Patent No. 5866371 includes a proximal portion 12, an intermediate portion 14, a sensor housing portion 15, and a distal end portion 16, in which a pressure sensor 34 is arranged in the inside of the sensor housing part 15 so as to be positioned in the vicinity of an opening 30 formed at the sensor housing part 15.

Moreover, a spiral cut pattern is formed in the intermediate portion 14 of the pressure-sensor guide wire 10, and an inner hypotube 20 is inserted into the intermediate portion 14 so as to be overlapped with the spiral cut pattern.

However, the pressure-sensor guide wire described in Japanese Patent No. 5866371 suffers from the following problem: flexibility obtained from the presence of the spiral cut pattern formed in the intermediate portion may eventually be impaired by the inner hypotube inserted into the intermediate portion, resulting in rupture when the pressure-sensor guide wire described in Japanese Patent No. 5866371 is curved to a large curvature.

Accordingly, an idea in that another cut pattern is formed in the inner hypotube in order to maintain flexibility has been proposed. However, this was found to suffer from the following problem: when the guide wire is curved, the edge of an outer slit may interfere with the edge of an inner slit or vice versa if the slits are formed arbitrarily. Therefore, the guide wire may become inoperable inside the body of a patient during the procedure.

Moreover, the pressure-sensor guide wire described in Japanese Patent No. 5866371 is configured such that the intermediate portion and the inner hypotube are separate entities. Disadvantageously, this may generate a gap between them during various operations of the guide wire, resulting in impaired torquability.

### SUMMARY

The disclosed embodiments have been devised to address the aforementioned problems. An object of the disclosed embodiments is to provide a medical tube which can be used in a medical instrument, including a guide wire, the medical tube having secure torquability, enhanced flexibility, and dispersed stress to prevent rupture without becoming inoperable even when curved.

As disclosed above, a medical tube of the disclosed embodiments includes a first hollow shaft including a proximal end portion and a distal end portion, the distal end portion being formed at the distal end side of the proximal end portion and having an outer diameter smaller than that of the proximal end portion and including a first slit formed on a surface thereof, the first slit extending in a first direction; and a second hollow shaft covering the outer periphery of the distal end portion of the first hollow shaft and including a second slit formed on a surface thereof, the second slit extending in a second direction crossing the first direction. This can allow for secured torquability, improved flexibility, and further dispersion of stress over the first hollow shaft and the second hollow shaft to prevent rupture of the medical tube without causing the medical tube to become inoperable even when curved.

The first and second slits may both extend spirally. This can provide an advantageous effect of simple manufacturing of the medical tube, in addition to the advantageous effect discussed above.

A spiral direction of the second slit formed on the second hollow shaft may be opposite to the spiral direction of the first slit formed on the first hollow shaft. Therefore, in addition to the advantageous effects discussed above, the first hollow shaft or the second hollow shaft can rotate in a tightening direction when a proximal end of the medical tube is rotated in either the clockwise or counter-clockwise direction, leading to improved torquability of the medical tube.

The second hollow shaft may include a hollow twisted wire, the hollow twisted wire including a single twisted elemental wire or multiple twisted elemental wires. This, in addition to the advantageous effects discussed above, can further improve flexibility of the distal end portion of the medical tube.

An outer diameter of the distal end of the second hollow shaft in a state where the second hollow shaft covers the outer periphery of the distal end portion of the first hollow shaft may be smaller than that of the proximal end portion of the first hollow shaft. This, in addition to the advantageous effects discussed above, can further improve flexibility of the distal end portion.

### BRIEF DESCRIPTION OF THE DRAWINGS

FIG. 1 shows a schematic diagram of a sensor guide wire including a medical tube according to the disclosed embodiments.

FIG. 2 shows an enlarged external view of a portion A of the medical tube shown in FIG. 1.

FIG. 3A shows an external view of an outer tube shaft (the second hollow shaft) of the medical tube shown in FIG. 1, and FIG. 3B shows a sectional view taken along line B-B shown in FIG. 3A.

FIG. 4A shows an external view of an inner tube shaft (the first hollow shaft) of the medical tube shown in FIG. 1, and FIG. 4B shows a sectional view taken along line C-C shown in FIG. 4B.

FIG. 5 shows a longitudinal sectional view of the medical tube shown in FIG. 1.

FIG. 6A shows an external view of an outer tube shaft (the second hollow shaft) of a medical tube according to the disclosed embodiments, and FIG. 6B shows a sectional view taken along line D-D shown in FIG. 6B.

FIG. 7 shows an external view of the medical tube shown in FIGS. 6A and 6B.

FIG. 8A shows an external view of an outer tube shaft (the second hollow shaft) of a medical tube according to the disclosed embodiments, and FIG. 8B shows a sectional view taken along line E-E shown in FIG. 8A.

FIG. 9 shows an external view of the medical tube shown in FIGS. 8A and 8B.

FIG. 10 shows an external view of a medical tube according to the disclosed embodiments.

FIG. 11 shows an external view of a medical tube according to the disclosed embodiments.

FIG. 12 shows an external view of a medical tube according to the disclosed embodiments.

### DETAILED DESCRIPTION OF EMBODIMENTS

Below, embodiments of the present invention will be described with reference to the drawings.

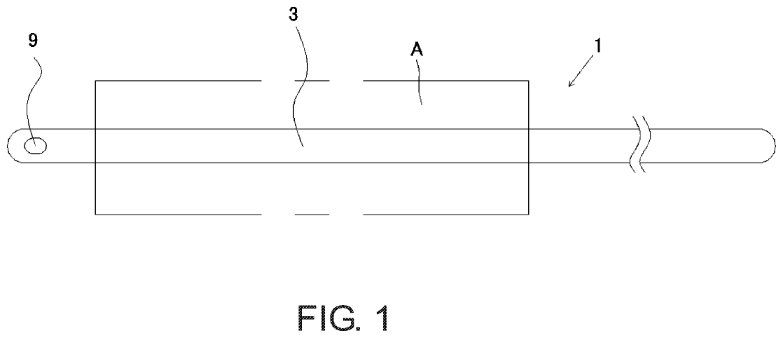

It is noted that the figures used for describing the disclosed embodiments may be drawn with exaggeration for clear understanding, and may not be scaled correctly. For example in FIG. 1, the shapes of the slits described below are significantly enlarged for clear understanding.

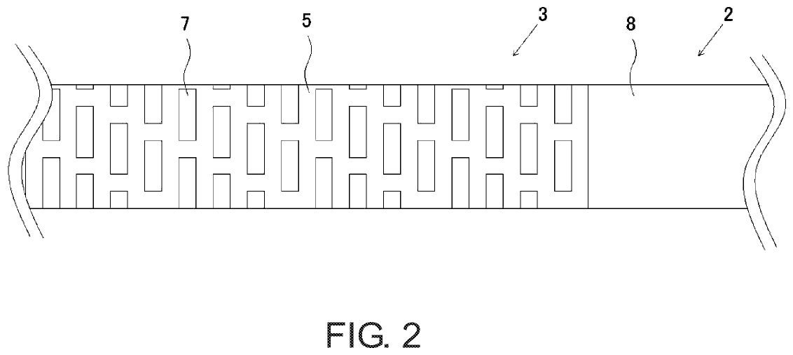

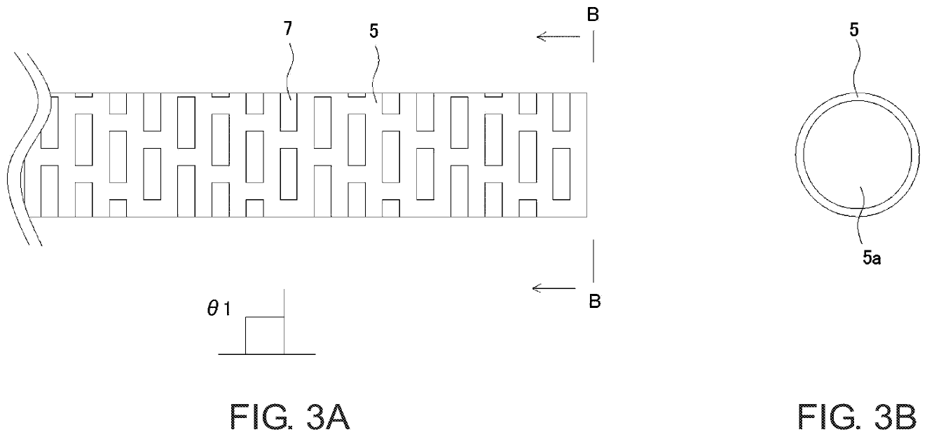

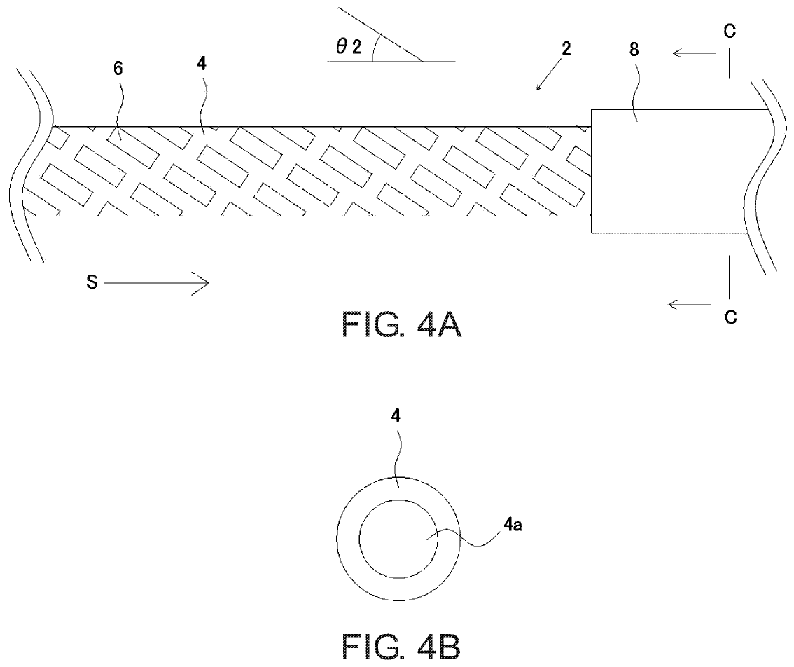

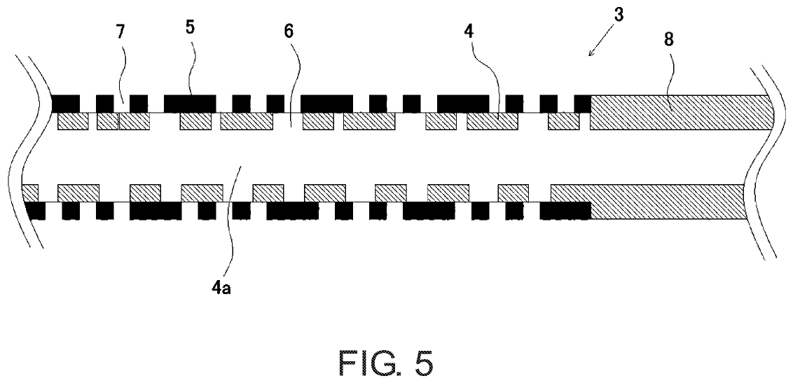

FIG. 1 shows a schematic diagram of a sensor guide wire including a medical tube according to the disclosed embodiments. FIG. 2 shows an enlarged external view of a portion A of the medical tube shown in FIG. 1. FIG. 3A shows an external view of an outer tube shaft (the second hollow shaft) of the medical tube shown in FIG. 1, and FIG. 3B shows a sectional view taken along line B-B shown in FIG. 3A. FIG. 4A shows an external view of an inner tube shaft (the first hollow shaft) of the medical tube shown in FIG. 1, and FIG. 4B shows a sectional view taken along line C-C shown in FIG. 4A. FIG. 5 shows a longitudinal sectional view of the medical tube shown in FIG. 1.

As shown in FIG. 1, a sensor guide wire 1 according to the disclosed embodiments includes a medical tube 3 having an elongated shape, an opening 9 formed at a distal end portion of the medical tube 3, and a sensor (not shown) arranged at the vicinity of the opening 9 in the inside of the medical tube 3. For example, the sensor guide wire 1 is inserted into a blood vessel when used. For example, a sensor and the like for measuring a blood temperature, a blood oxygen level, or a blood pressure may be used as a sensor in the sensor guide wire.

As shown in FIGS. 2 to 5, the medical tube 3 includes an inner tube shaft 2 (which corresponds to a “first hollow shaft”) including a proximal end portion 8 and a distal end portion 4, the distal end portion 4 being formed at a distal end side of the proximal end portion 8; and an outer tube shaft 5 (which corresponds to a “second hollow shaft”) inserted onto an outer periphery of the distal end portion 4 of the inner tube shaft 2 in an S direction (see FIG. 4A).

The inner tube shaft 2 is an elongated hollow cylindrical member, and includes the proximal end portion 8 and the distal end portion 4, the distal end portion 4 being formed at the distal end side of the proximal end portion 8. The outer diameter of the distal end portion 4 is smaller than that of the proximal end portion 8, and a plurality of slits 6 (each of which corresponds to a “first slit”) are formed on a surface of the distal end portion 4 spirally in the clockwise direction toward the distal end. Further, the inner tube shaft 2 has a lumen 4a in communication with the proximal end portion 8 and the distal end portion 4.

The direction in which the slits 6 extend is a θ2 direction (which corresponds to a “first direction”) relative to the longitudinal direction of the medical tube 3 as shown in FIG. 4A.

The outer tube shaft 5 is an elongated hollow cylindrical member as in the inner tube shaft 2, and has an outer diameter identical to that of the proximal end portion 8 of the inner tube shaft 2. Further, the outer tube shaft 5 includes a plurality of slits 7 (each of which corresponds to a “second slit”) formed on a surface thereof, the slits 7 extending in a θ1 direction (which corresponds to a “second direction”) perpendicular to the longitudinal direction of the medical tube 3. Moreover, the outer tube shaft 5 has a lumen 5a into which the distal end portion 4 of the inner tube shaft 2 can be inserted.

There is no particular limitation for the materials of the inner tube shaft 2 and the outer tube shaft 5 as long as they are flexible materials having biocompatibility, for example, metals or metal compounds such as stainless steel, nickel-titanium alloys, tungsten, gold, platinum, iridium, cobalt-chromium alloys; or polyolefins such as polyethylenes, polypropylenes, ethylene-propylene copolymers, ethylene-vinyl acetate copolymers; thermoplastic resins such as soft polyvinyl chlorides; various rubbers such as silicone rubber and latex rubber; various elastomers such as polyurethane elastomers, polyamide elastomers, and polyester elastomers; crystalline plastics such as polyamides, crystalline polyethylenes, and crystalline polypropylenes.

The medical tube 3 includes the inner tube shaft 2 including the proximal end portion 8 and the distal end portion 4, the distal end portion 4 being formed at the distal end side of the proximal end portion 8 and having an outer diameter smaller than that of the proximal end portion 8 and including the slits 6 formed on a surface thereof, the slits 6 extending in the θ2 direction relative to the longitudinal direction of the medical tube 3; and the outer tube shaft 5 covering the outer periphery of the distal end portion 4 of the inner tube shaft 2 and including the slits 7 formed on a surface thereof, the slits 7 extending in the θ1 second direction crossing the θ2 direction. Therefore, torquability can be secured by the integral formation of the proximal end portion 8 and the distal end portion 4 of the inner tube shaft 2. Further, flexibility can be improved by the presence of the slits 6 and the slits 7 formed on the distal end portion 4 of the inner tube shaft 2 and the outer tube shaft 5. Moreover, the direction in which the slits 6 extend is configured to cross the direction in which the slits 7 extend. This can disperse stress over the inner tube shaft 2 and the outer tube shaft 5 without causing the medical tube 3 to become inoperable even when the medical tube 3 is curved, thereby preventing rupture of the medical tube 3.

As described above, the slits 6 are configured so as to extend in the 02 direction relative to the longitudinal direction of the medical tube 3, and the slits 7 are configured so as to extend in the θ1 direction perpendicular to the longitudinal direction of the medical tube 3, but the configuration shall not be limited to this. Similar effects can be obtained as long as the slits 6 and the slits 7 are formed in directions where they cross each other.

For example, the slits 6 may be configured so as to extend in a clockwise or counter-clockwise first direction relative to the longitudinal direction of the medical tube 3, and the slits 7 may be configured so as to extend in the θ2 direction relative to the longitudinal direction of the medical tube 3.

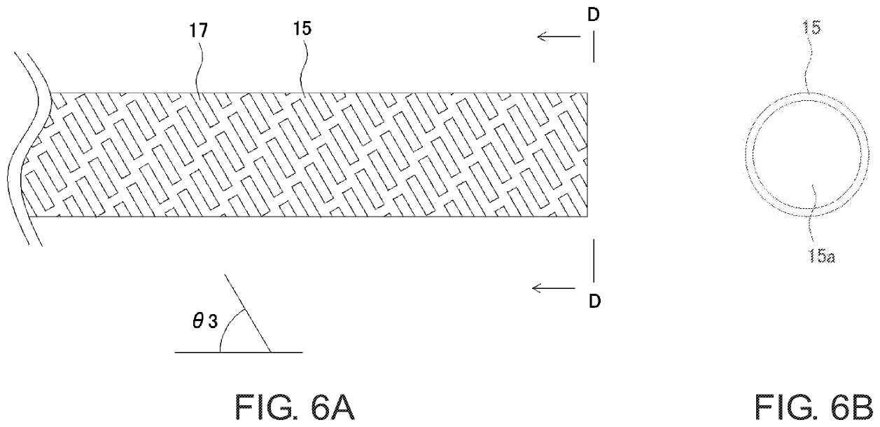

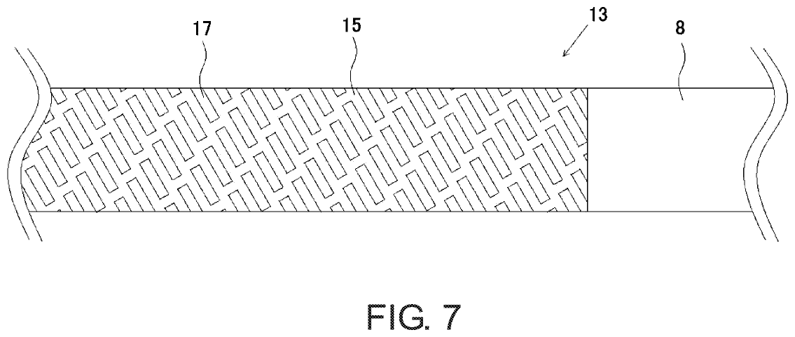

The sensor guide wire shown in FIGS. 6A-7 has an overall structure similar to that shown in FIG. 1 and an inner tube shaft 2 similar to that shown in FIG. 4A. Therefore, their descriptions are omitted. The same symbols are assigned to common portions, and their descriptions are omitted.

FIG. 6A shows an external view of an outer tube shaft (the second hollow shaft) of a medical tube of the disclosed embodiments, and FIG. 6B shows a sectional view taken along line D-D shown in FIG. 6A. FIG. 7 shows an external view of the medical tube shown in FIGS. 6A and 6B.

As shown in FIGS. 6A-7, a medical tube 13 includes the inner tube shaft 2 (which corresponds to the “first hollow shaft”) including the proximal end portion 8 and the distal end portion 4, the distal end portion 4 being formed at the distal end side of the proximal end portion 8; and an outer tube shaft 15 (which corresponds to the “second hollow shaft”) inserted onto the outer periphery of the distal end portion 4 of the inner tube shaft 2.

The outer tube shaft 15 is an elongated hollow cylindrical member as in the inner tube shaft 2, and has an outer diameter identical to that of the proximal end portion 8 of the inner tube shaft 2. Further, the outer tube shaft 15 includes a plurality of slits 17 (each of which corresponds to the “second slit”) formed on a surface thereof spirally in the clockwise direction toward the distal end.

It is noted that the direction in which the slits 17 extend is a —03 direction (which corresponds to the “second direction”: see FIG. 6A) relative to the longitudinal direction of the medical tube 13, and is different from the θ2 direction of the slits 6. Further, the outer tube shaft 15 has a lumen 15a into which the distal end portion 4 of the inner tube shaft 2 can be inserted.

Moreover, the outer tube shaft 15 may be made of a similar material as those of the inner tube shaft 2 and the outer tube shaft 5.

In the medical tube 13, the slits 6 formed on the inner tube shaft 2 and the slits 17 formed on the outer tube shaft 15 are both configured so as to extend spirally. Therefore, the medical tube 13 can be manufactured easily. Torquability can be secured by the integral formation of the proximal end portion 8 and the distal end portion 4 of the inner tube shaft 2. Further, flexibility can be improved by the presence of the slits 6 and the slits 17 formed on the distal end portion 4 of the inner tube shaft 2 and the outer tube shaft 15. Moreover, the direction in which the slits 6 extend is configured to cross the direction in which the slits 17 extend. This can disperse stress over the inner tube shaft 2 and the outer tube shaft 15 without causing the medical tube 13 to become inoperable even when the medical tube 13 is curved, thereby preventing rupture of the medical tube 13.

As described above, the slits 6 are configured so as to extend in the 02 direction relative to the longitudinal direction of the medical tube 13, and the slits 17 are configured so as to extend in the θ3 direction relative to the longitudinal direction of the medical tube 13, but the configuration shall not be limited to this. Similar effects can be obtained as long as the slits 6 and the slits 17 are formed spirally in directions where they cross each other.

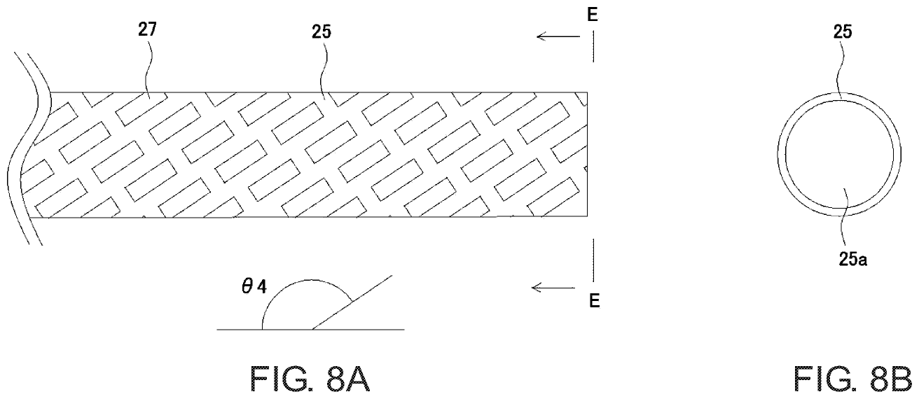

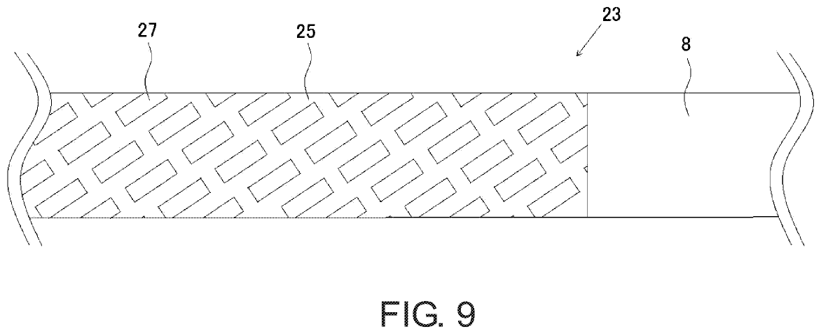

The sensor guide wire shown in FIGS. 8A-9 has an overall structure similar to that shown in FIG. 1 and an inner tube shaft 2 similar to that shown in FIG. 4A. Therefore, their descriptions will be omitted. The same symbols are assigned to common portions, and their descriptions are omitted.

FIG. 8A shows an external view of an outer tube shaft (the second hollow shaft) of a medical tube according to the disclosed embodiments, and FIG. 8B shows a sectional view along line E-E shown in FIG. 8A. FIG. 9 shows an external view of the medical tube shown in FIGS. 8A and 8B.

As shown in FIGS. 8A-9, a medical tube 23 includes the inner tube shaft 2 (which corresponds to the “first hollow shaft”) including the proximal end portion 8 and the distal end portion 4, the distal end portion 4 being formed at the distal end side of the proximal end portion 8; and an outer tube shaft 25 (which corresponds to the “second hollow shaft”) inserted onto the outer periphery of the distal end portion 4 of the inner tube shaft 2.

The outer tube shaft 25 is an elongated hollow cylindrical member as in the inner tube shaft 2, and has an outer diameter identical to that of the proximal end portion 8 of the inner tube shaft 2. Further, the outer tube shaft 25 includes a plurality of slits 27 (each of which corresponds to the “second slit”) formed on a surface thereof spirally in the counter-clockwise direction toward the distal end.

It is noted that the direction in which the slits 27 extend is a θ4 direction (which corresponds to the “second direction”: see FIG. 8A) relative to the longitudinal direction of the medical tube 23, and is spirally opposite to the θ2 direction of the slits 6. Further, the outer tube shaft 25 has a lumen 25a into which the distal end portion 4 of the inner tube shaft 2 can be inserted.

Moreover, the outer tube shaft 25 may be made of a similar material as those of the outer tube shaft 5 and the outer tube shaft 15.

In the medical tube 23, the spiral direction of the slits 27 formed on the outer tube shaft 25 is opposite to the spiral direction of the slits 6 formed on the inner tube shaft 2. Therefore, the outer tube shaft 25 or the inner tube shaft 2 can rotate in a tightening direction when a proximal end of the medical tube 23 is rotated in either the clockwise or counter-clockwise direction, leading to improved torquability of the medical tube 23.

Further, torquability can be secured by the integral formation of the proximal end portion 8 and the distal end portion 4 of the inner tube shaft 2. Moreover, flexibility can be improved by the presence of the slits 6 and the slits 27 formed on the distal end portion 4 of the inner tube shaft 2 and the outer tube shaft 25. Furthermore, the direction in which the slits 6 extend is configured to cross the direction in which the slits 27 extend. This can disperse stress over the inner tube shaft 2 and the outer tube shaft 25 without causing the medical tube 23 to become inoperable even when the medical tube 23 is curved, thereby preventing rupture of the medical tube 23.

As described above, the slits 6 are configured so as to extend in the 02 direction relative to the longitudinal direction of the medical tube 23, and the slits 27 are configured so as to extend in the θ4 direction relative to the longitudinal direction of the medical tube 23, but the configuration shall not be limited to this. Similar effects can be obtained as long as the slits 6 and the slits 27 are formed in spirally opposite directions.

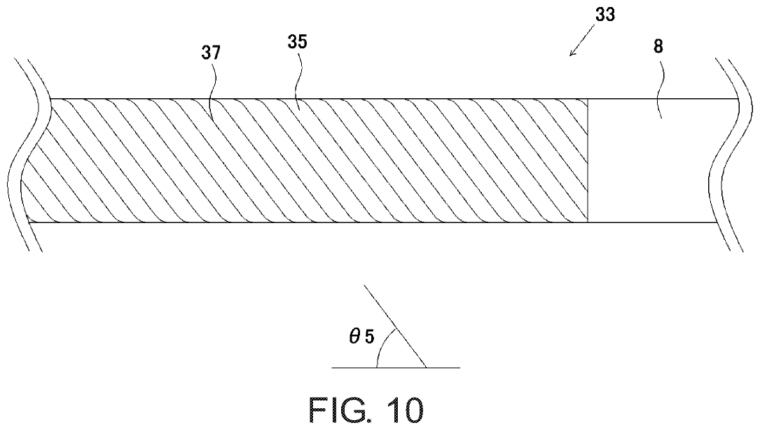

The sensor guide wire shown in FIG. 10 has an overall structure similar to that shown in FIG. 1 and an inner tube shaft 2 similar to that shown in FIG. 4A. Therefore, their descriptions will be omitted. The same symbols are assigned to common portions, and their descriptions are omitted.

FIG. 10 shows an external view of a medical tube according to the disclosed embodiments.

As shown in FIG. 10, a medical tube 33 includes the inner tube shaft 2 (which corresponds to the “first hollow shaft”) including the proximal end portion 8 and the distal end portion 4, the distal end portion 4 being formed at the distal end side of the proximal end portion 8; and a hollow twisted wire 35 (also called a wire coil or coiled body, which corresponds to the “second hollow shaft”) including a single twisted element wire or multiple twisted element wires wound around the outer periphery of the distal end portion 4 of the inner tube shaft 2.

The outer diameter of the hollow twisted wire 35 is identical to that of the proximal end portion 8 of the inner tube shaft 2, and a plurality of depressed portions 37 (each of which corresponds to the “second slit”) are formed spirally in the clockwise direction toward the distal end on a surface of the hollow twisted wire 35.

It is noted that the direction in which the slits 37 extend is a θ5 direction (which corresponds to the “second direction”: see FIG. 10) relative to the longitudinal direction of the medical tube 33, and is different from the θ2 direction of the slits 6. Further, the hollow twisted wire 35 has a lumen (not shown) into which the distal end portion 4 of the inner tube shaft 2 can be inserted.

Moreover, the hollow twisted wire 35 may be made of a similar material as those of the inner tube shaft 2 and the outer tube shaft 5.

In the medical tube 33, the hollow twisted wire 35 is formed with a single twisted element wire or multiple twisted element wires. This can further improve flexibility of the distal end portion 4 of the medical tube 33.

Further, torquability can be secured by the integral formation of the proximal end portion 8 and the distal end portion 4 of the inner tube shaft 2. Moreover, flexibility can be improved by the presence of the slits 6 and the depressed portions 37 formed on the distal end portion 4 of the inner tube shaft 2 and the hollow twisted wire 35. Furthermore, the direction in which the slits 6 extend is configured to cross the direction in which the depressed portions 37 extend. This can disperse stress over the inner tube shaft 2 and the hollow twisted wire 35 without causing the medical tube 33 to become inoperable even when the medical tube 33 is curved, thereby preventing rupture of the medical tube 33.

As described above, the slits 6 are configured so as to extend in the 02 direction relative to the longitudinal direction of the medical tube 33, and the depressed portions 37 are configured so as to extend in the θ5 direction relative to the longitudinal direction of the medical tube 33, but the configuration shall not be limited to this. Similar effects can be obtained as long as the slits 6 and the depressed portions 37 are formed spirally in directions where they cross each other.

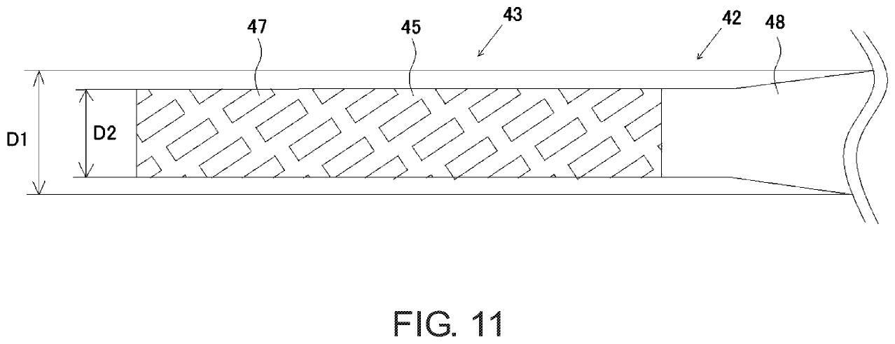

The sensor guide wire shown in FIG. 11 has an overall structure similar to that shown in FIG. 1. Therefore, descriptions thereof will be omitted. The same symbols are assigned to common portions, and their descriptions are omitted.

FIG. 11 shows an external view of a medical tube according to the disclosed embodiments.

As shown in FIG. 11, a medical tube 43 includes an inner tube shaft 42 (which corresponds to the “first hollow shaft”) including a proximal end portion 48 and a distal end portion (not shown), the distal end portion being formed at the distal end side of the proximal end portion 48; and an outer tube shaft 45 (which corresponds to the “second hollow shaft”) inserted onto the outer periphery of the distal end portion of the inner tube shaft 42.

The inner tube shaft 42 is an elongated hollow cylindrical member, and includes the proximal end portion 48 and the distal end portion (not shown), the distal end portion being formed at the distal end side of the proximal end portion 48. The distal end portion has an outer diameter smaller than that of the proximal end portion 48, and a plurality of slits 46 (not shown, each of which corresponds to the “first slit” and is in a form similar to the slits 6) that are formed spirally in the clockwise direction toward the distal end on a surface of the distal end portion. Further, the inner tube shaft 42 has a lumen in communication with the proximal end portion 48 and the distal end portion thereof.

The direction in which the slits 46 extend is the θ2 direction (which corresponds to the “first direction”: see FIG. 4A).

The outer tube shaft 45 is an elongated hollow cylindrical member as in the inner tube shaft 42, and an outer diameter D2 thereof is smaller than an outer diameter D1 of the proximal end portion 48 of the inner tube shaft 42. Further, the outer tube shaft 45 includes a plurality of slits 47 (each of which corresponds to the “second slit”) formed on a surface thereof spirally in the counter-clockwise direction toward the distal end.

It is noted that the direction in which the slits 47 extend is the 04 direction (which corresponds to the “second direction”: see FIG. 8A) relative to the longitudinal direction of the medical tube 43, and is spirally opposite to the θ2 direction of the slits 6. Moreover, the outer tube shaft 45 has a lumen (not shown) into which the distal end portion of the inner tube shaft 42 can be inserted.

Moreover, the inner tube shaft 42 and the outer tube shaft 45 may be made of similar materials as those of the inner tube shaft 2 and the outer tube shaft 5.

In the medical tube 43, the outer diameter of the distal end of the outer tube shaft 45 in a state where the outer tube shaft 45 covers the outer periphery of the distal end portion of the inner tube shaft 42 is smaller than that of the proximal end portion of the inner tube shaft 42. This can further improve flexibility of the distal end portion of the medical tube 43.

Further, torquability can be secured by the integral formation of the proximal end portion 48 and the distal end portion of the inner tube shaft 42. Moreover, flexibility can be improved by the presence of the slits 46 and the slits 47 formed on the distal end portion of the inner tube shaft 42 and the outer tube shaft 45. Furthermore, the direction in which the slits 46 extend is configured to cross the direction in which the slits 47 extend. This can disperse stress over the inner tube shaft 42 and the outer tube shaft 45 without causing the medical tube 43 to become inoperable even when the medical tube 43 is curved, thereby preventing rupture of the medical tube 43.

As described above, the slits 46 are configured so as to extend in the 02 direction relative to the longitudinal direction of the medical tube 43, and the slits 47 are configured so as to extend in the θ4 direction relative to the longitudinal direction of the medical tube 43, but the configuration shall not be limited to this. Similar effects can be obtained as long as the slits 46 and the slits 47 are formed in directions where they cross each other.

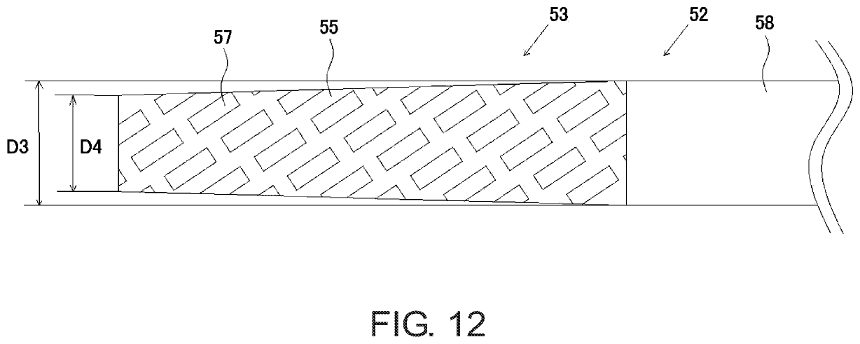

The sensor guide wire shown in FIG. 12 has an overall structure similar to that shown in FIG. 1. Therefore, descriptions thereof will be omitted. The same symbols are assigned to common portions, and their descriptions are omitted.

FIG. 12 shows an external view of a medical tube according to the disclosed embodiments.

As shown in FIG. 12, a medical tube 53 includes an inner tube shaft 52 (which corresponds to the “first hollow shaft”) including a proximal end portion 58 and a distal end portion (not shown), the distal end portion being formed at the distal end side of the proximal end portion 58; and an outer tube shaft 55 (which corresponds to the “second hollow shaft”) inserted onto the outer periphery of the distal end portion of the inner tube shaft 52.

The inner tube shaft 52 is an elongated hollow cylindrical member, and includes the proximal end portion 58 and the distal end portion (not shown), the distal end portion being formed at the distal end side of the proximal end portion 58. The distal end portion has an outer diameter smaller than that of the proximal end portion 58, and a plurality of slits 56 (not shown, each of which corresponds to the “first slit” and is in a form similar to the slits 6) that are formed spirally in the clockwise direction toward the distal end on a surface of the distal end portion. Further, the inner tube shaft 52 has a lumen in communication with the proximal end portion 58 and the distal end portion thereof.

The direction in which the slits 56 extend is the θ2 direction (which corresponds to the “first direction”: see FIG. 4A).

The outer tube shaft 55, which is an elongated hollow member, has an external shape tapered toward the distal end, and an outer diameter D4 of the distal end thereof is smaller than an outer diameter D3 of the proximal end portion 58 of the inner tube shaft 52. Further, the outer tube shaft 55 includes a plurality of slits 57 (each of which corresponds to the “second slit”) formed on a surface thereof spirally in the counter-clockwise direction toward the distal end.

It is noted that the direction in which the slits 57 extend is the 04 direction (which corresponds to the “second direction”: see FIG. 8A) relative to the longitudinal direction of the medical tube 53, and is spirally opposite to the θ2 direction of the slits 6. Moreover, the outer tube shaft 55 has a lumen (not shown) into which the distal end portion of the inner tube shaft 52 can be inserted.

Moreover, the inner tube shaft 52 and the outer tube shaft 55 may be made of similar materials as those of the inner tube shaft 2 and the outer tube shaft 5.

In the medical tube 53, the outer diameter of the distal end of the outer tube shaft 55 in a state where the outer tube shaft 55 covers the outer periphery of the distal end portion of the inner tube shaft 52 is smaller than that of the proximal end portion of the inner tube shaft 52. This can further improve flexibility of the distal end portion of the medical tube 53.

Further, torquability can be secured by the integral formation of the proximal end portion 58 and the distal end portion of the inner tube shaft 52. Moreover, flexibility can be improved by the presence of the slits 56 and the slits 57 formed on the distal end portion of the inner tube shaft 52 and the outer tube shaft 55. Furthermore, the direction in which the slits 56 extend is configured to cross the direction in which the slits 57 extend. This can disperse stress over the inner tube shaft 52 and the outer tube shaft 55 without causing the medical tube 53 to become inoperable even when the medical tube 53 is curved, thereby preventing rupture of the medical tube 53.

As described above, the slits 56 are configured so as to extend in the 02 direction relative to the longitudinal direction of the medical tube 53, and the slits 57 are configured so as to extend in the θ4 direction relative to the longitudinal direction of the medical tube 53, but the configuration shall not be limited to this. Similar effects can be obtained as long as the slits 56 and the slits 57 are formed in directions where they cross each other.

Medical tubes according to various embodiments of the present invention are described above, but the present invention shall not be limited to these. Various alterations may be made without departing from the spirit of the present invention.

For example, the shapes of the slits in the drawings are drawn with exaggeration for illustrative purposes, but the shapes shall not be limited to these. The width and length of a slit may be appropriately selected.

Further, the wire diameter of an elemental wire for the hollow twisted wire 35 shown in FIG. 10 may also be selected appropriately.

Moreover, a plurality of slits is configured so as to extend spirally on a surface of the outer peripheral of an inner tube shaft and/or an outer tube shaft, but the configuration shall not be limited to this. For example, a configuration (spiral cut) may be used in which a single slit is formed so as to extend spirally.

1. A medical tube comprising:
a first hollow shaft including a proximal end portion and a distal end portion, the distal end portion having an outer diameter smaller than an outer diameter of the proximal end portion and including a first slit formed on an outer surface of the distal end portion, the first slit extending in a first direction; and
a second hollow shaft covering the outer surface of the distal end portion of the first hollow shaft and including a second slit formed on a surface of the second hollow shaft, the second slit extending in a second direction crossing the first direction.

2. The medical tube according to claim 1, wherein the first slit and the second slit extend spirally.

3. The medical tube according to claim 2, wherein a spiral direction of the second slit is opposite to a spiral direction of the first slit.

4. The medical tube according to claim 2, wherein the second hollow shaft comprises a coiled body, the coiled body comprising at least one element wire.

5. The medical tube according to claim 3, wherein the second hollow shaft comprises a coiled body, the coiled body comprising at least one element wire.

6. The medical tube according to claim 1, wherein an outer diameter of a distal end of the second hollow shaft is smaller than the outer diameter of the proximal end portion of the first hollow shaft.

7. The medical tube according to claim 2, wherein an outer diameter of a distal end of the second hollow shaft is smaller than the outer diameter of the proximal end portion of the first hollow shaft.

8. The medical tube according to claim 3, wherein an outer diameter of a distal end of the second hollow shaft is smaller than the outer diameter of the proximal end portion of the first hollow shaft.

9. The medical tube according to claim 4, wherein an outer diameter of a distal end of the second hollow shaft is smaller than the outer diameter of the proximal end portion of the first hollow shaft.

10. The medical tube according to claim 1, wherein an outer diameter of the second hollow shaft is tapered from a proximal end of the second hollow shaft toward a distal end of the second hollow shaft.

11. The medical tube according to claim 1, wherein an outer diameter of the first hollow shaft is tapered from a proximal end of the first hollow shaft toward a distal end of the first hollow shaft.

12. The medical tube according to claim 1, wherein the first hollow shaft includes a plurality of the first slit formed on the outer surface of the distal end portion.

13. The medical tube according to claim 1, wherein the first hollow shaft includes only a single one of the first slit formed on the outer surface of the distal end portion.

14. The medical tube according to claim 1, wherein the second hollow shaft includes a plurality of the second slit formed on the outer surface of the second hollow shaft.

15. The medical tube according to claim 1, wherein the second hollow shaft includes only a single one of the second slit formed on the outer surface of the second hollow shaft.
